(19) United States
(12) Patent Application Publication
(10) Pub. No.: US 2005/0009717 A1
(43) Pub. Date: Jan. 13, 2005

(54) Foaming make-up removing cleansing compositions

(76) Inventors: Elvin R. Lukenbach, Flemington, NJ (US); Catherine Coret, Robbinsville, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/862,634

(22) Filed: Jun. 7, 2004

Related U.S. Application Data
(63) Continuation-in-part of application No. 09/604,449, filed on Jun. 27, 2000.
(60) Provisional application No. 60/141,927, filed on Jul. 1, 1999.

(30) Foreign Application Priority Data
Jun. 23, 2000 (WO) PCT/US00/17431

(51) Int. Cl. A61K 7/02

(52) U.S. Cl. 510/136

(57) ABSTRACT

The present invention relates to cleansing compositions suitable for use in personal cleansing applications, and in particular make-up removal applications, which impart superior cleansing properties and are comprised of a liquid, water-insoluble propoxylated fatty alcohol, water dispersible components, water, and foaming surfactants. Also disclosed are compositions for effectively depositing various benefit agents into and onto the skin.

### CROSS-REFERENCE TO RELATED APPLICATIONS

This Application is a continuation-in-part application of U.S. patent application Ser. No. 09/604,449 filed on Jun. 27, 2000, which claims priority from U.S. Provisional Patent Application No. 60/141,927, filed Jul. 1, 1999, the disclosures of which are incorporated herein by reference in their entirety.

### BACKGROUND OF THE INVENTION

1. Field of the Invention

This invention relates to foaming cleansing compositions suitable for use in personal cleansing applications, and in particular make-up removal applications, which impart superior cleansing properties. This invention is further related to a composition for effectively delivering and/or depositing various benefit agents into and onto the skin.

2. Description of the Prior Art

Various types of cosmetics such as make-up, e.g. lipstick, mascara, foundation, and the like, leave an oil-containing residue on the skin surface that cannot be removed easily by facial cleansers containing conventional soaps. One reason is that such soaps are unable to effectively emulsify or solubilize such oils, which is why many make-up remover compositions have included an oil base as a major component. For example, European Patent No. 370856 discloses a non-foaming makeup remover system comprised of a surfactant-containing water phase that remains physically separated from a cosmetic oil-containing oil phase unless shaken. Disadvantageously, such oil-containing removers also suffer from a tendency to deposit an oily residue or film on the user's skin.

Various attempts have been made to produce stable, oil-free makeup removers. For example, U.S. Pat. No. 5,217,641 discloses an oil-free, stable, non-irritating, single-phase makeup remover comprised of 50 percent to 98 percent of cyclomethicone along with a mixture of esters. However, not only is it economically disadvantageous to use such a large amount of cyclomethicones, but because of their highly volatile nature, cyclomethicones cannot be packaged easily using conventional cosmetic packaging.

One reason that make-up cannot be removed by conventional soaps is the fact that such soaps are incapable of removing the binders in the make-up. These binders tend to increase the make-up's resistance against sebum and water as well as its overall adhesiveness to the skin. In addition, various polymers, which are similarly difficult to remove, are also employed in hair cosmetics for the purpose of protecting the hair or providing the hair with body.

It would be desirable to have a stable, economically-feasible composition that could not only effectively remove the residue from sebum as well as the residue from make-up and hair-protecting agents, but also impart a pleasant, non-oily “after-feel” to the skin and hair. It would further be desirable to create such a composition that is capable of depositing various active agents into and onto the skin.

### SUMMARY OF THE INVENTION

In accordance with this invention, there is provided a cleansing composition comprising
a. a water dispersible component; b. a liquid, water-insoluble propoxylated fatty alcohol; and c. water.

Another embodiment of the present invention is directed to a method for depositing benefit agents into and onto the skin comprised of: topically applying an effective amount of the benefit agent with a composition comprised of a water dispersible component; a liquid, water-insoluble propoxylated fatty alcohol; and water to a desired location.

The invention will be more fully understood and further advantages will become apparent when reference is made to the following detailed description of the invention.

### DETAILED DESCRIPTION OF PREFERRED EMBODIMENTS

In one embodiment of the present invention, the cleansing composition may suitably comprise, consist of, or consist essentially of, based upon the total weight of the cleansing composition, from about 10 percent to about 35 percent, and preferably from about 10 percent to about 20 percent of a water dispersible component; and from about 30 percent to about 80 percent, and preferably from 55 percent to about 65 percent of a liquid, water-insoluble propoxylated fatty alcohol. The balance of the cleansing composition comprises water and optionally, a foaming surfactant.

The first component of the cleansing composition of the present invention is a water dispersible component, which is preferably a water soluble solvent. As used herein, the term “water dispersible component” shall mean a material that produces a uniform, clear or hazy, mixture when combined with at least a weight equivalent of water. Examples of suitable water dispersible components nonexclusively include polyethylene glycol 400, hexylene glycol, propylene glycol, polypropylene glycol-10 methylglucose ether, ethoxydiglycol, polyethylene glycol-6 caprylic/capric glyceride, ethylene glycol monobutyl ether, polyethylene glycol-8 caprylic/capric glycerides, 3-methoxy-3-methyl-1-butanol, dimethyl isosorbide, and mixtures thereof. Most preferred water dispersible components include hexylene glycol, dimethyl isosorbide, polyethylene glycol-6 caprylic/capric glyceride, and mixtures thereof.

The second component of the cleansing composition of the present invention is a liquid, water-insoluble propoxylated fatty alcohol, RO(CH₂CHOCH₃)n, in which the “fatty” group R contains from about 12 to about 22 carbon atoms. The fatty group R may be straight chain or branched, saturated or unsaturated. The degree of propoxylation may range from about 1 to about 100, for example, from about 5 to 50 or from about 10 to about 20. A preferred liquid, water-insoluble propoxylated fatty alcohol is polypropylene glycol 15 stearyl ether commercially available from Uniqema as ARLAMOL E.

The liquid, water-insoluble propoxylated fatty alcohol may be combined with a liquid ester. Suitable liquid esters include:
a) a branched C₅to C₂₂ alkyl alcohol ester of an aromatic acid;
b) a straight-chained or branched C₅ to C₂₂ alkyl acid esters of optionally ethyoxylated/propoxylated polyols having from about 3 carbon atoms to about 7 carbon atoms;
c) branched C₅to C₂₂ alkyl alcohol esters of branched polyacids;
d) branched or straight-chained C₅ to C₂₂ alkyl acid esters of branched and/or unsaturated C₅to C₂₂ alkyl alcohols;
e) branched or unsaturated C₅ to C₂₂ alkyl alcohol esters of an acid selected from the group consisting of adipic acid, succinic acid, maleic acid, sebacic acid, and mixtures thereof
f) polyether interrupted fatty acid esters;
g) benzoic acid ester of heterogeneous alcohols having from about 8 carbon atoms to about 22 carbon atoms; and
h) mixtures thereof,
with straight-chained or branched C₅ to C₂₂ alkyl acid esters of optionally ethyoxylated/propoxylated polyols, benzoic acid esters of heterogeneous alcohols, and mixtures thereof being particularly preferred.

Suitable branched C₅ to C₂₂ alkyl alcohol esters of an aromatic acid include those wherein the aromatic acid is benzoic acid. Preferably, the alcohol of this ester is either branched or unsaturated, and may be either a primary alcohol or a secondary alcohol with the former being preferred. Optionally, the aromatic acid may be substituted with hydroxy or alkyl groups having from about 1 carbon atom to about 4 carbon atoms. Specific examples of these esters nonexclusively include, butyloctyl salicylate; hexyldecyl benzoate; and butyloctyl benzoate, which are all available from C.P. Hall Co. under the tradename, “HallStar;” and mixtures thereof, with a mixture of hexyldecyl benzoate and butyloctyl benzoate being particularly preferred.

Another suitable ester includes a straight-chained or branched C₅ to C₂₂ alkyl acid ester of optionally ethyoxylated/propoxylated polyols, wherein the polyols contain from about 3 carbon atoms to about 7 carbon atoms. Preferably, if the polyol creates a branching point, then the acid group may be straight-chained. Suitable acids used to form these esters typically have from about 8 carbon atoms to about 22 carbon atoms, and preferably from about 8 carbon atoms to about 18 carbon atoms, and most preferably from about 8 carbon atoms to about 12 carbon atoms, and are either saturated or unsaturated, with octanoic acid, capric acid, and mixtures thereof being preferred. Such suitable acids are either straight-chained or branched, and are preferably aliphatic. Suitable polyols used to form these esters typically have from about 3 carbon atoms to about 30 carbon atoms, and preferably from about 3 carbon atoms to about 7 carbon atoms. Examples of such suitable polyols nonexclusively include neopentyl alcohol; polyglycerol, e.g. diglycerol, triglycerol, hexaglycerol, and decaglycerol, wherein the polyglycerol may contain from about 2 to about 10 glycerol groups; glycerin; sorbitan; methyl glucose; trimethylolpropane; and mixtures thereof. Neopentyl alcohol, glycerin, trimethylolpropane, and mixtures thereof are the preferred polyols. Examples of suitable esters nonexclusively include pentaerythritol tetraoctanoate; trimethylolpropane trioctanoate; trioctanoin; pentaerythrityl tetrapelargonate; sorbitan trioleate; caprylic/capric triglyceride; neopentyl alcohol tetraoctanoate, and mixtures thereof, with caprylic/capric triglyceride; pentaerythritol tetraoctanoate; trimethylolpropane trioctanoate; and pentaerythrityl tetrapelargonate being more preferred.

Another suitable ester includes the branched C₅ to C₂₂ alkyl alcohol esters of branched polyacids such as the tri-esters, tetra-esters, penta-esters, and mixtures thereof. An example of such a polyacid is citric acid. Suitable alkyl alcohols for creating these esters are optionally substituted, e.g., ethoxylated or propoxylated, contain from about 3 carbon atoms to about 22 carbon atoms, and preferably from about 3 carbon atoms to about 8 carbon atoms, and are either straight-chained or branched, with the branching being preferred. These alcohols may either be primary or secondary, and may either be saturated or unsaturated, with saturated being preferred for stability reasons. Specific examples of suitable esters nonexclusively include trioctyidodecyl citrate; triisopropylcitrate; and mixtures thereof.

Another suitable ester includes the branched or straight-chained C₅ to C₂₂ alkyl acid esters of branched or unsaturated alkyl alcohols wherein the alkyl group of the alcohol has from about 1 carbon atoms to about 18 carbon atoms, and preferably from about 4 carbon atoms to about 10 carbon atoms, provided that the total number of carbon atoms in the ester is at least about 8. Suitable acids for use in making these esters typically have from about 2 carbon atoms to about 22 carbon atoms, and preferably from about 5 carbon atoms to about 10 carbon atoms. However, if the number of acid carbon atoms exceeds the number of carbon atoms in the alcohol, then the acid preferably contains from about 8 carbon atoms to about 18 carbon atoms and the alcohol preferably contains from about 1 carbon atom to about 8 carbon atoms. If the number of acid carbon atoms is less than the number of carbon atoms in the alcohol, then the acid preferably contains from about 2 carbon atoms to about 8 carbon atoms and the alcohol preferably contains from about 8 carbon atoms to about 18 carbon atoms. Preferably, either: 1) the alcohol group or the acid group has branching and/or unsaturation, i.e. both the alcohol and the acid are not straight-chained; or 2) the ester possesses an asymmetrical alkyl distribution. By “asymmetrical alkyl distribution,” it is meant that the ester is made from, for example, a short chain alcohol, i.e. having from about 1 carbon atom to about 8 carbon atoms, and a long chain acid, i.e., having greater than about 8 carbon atoms, such as, e.g. butyl stearate, or less preferably the ester is made from, a long chain alcohol, i.e. having greater than about 8 carbon atoms, and a short chain acid, i.e. having from about 1 carbon atom to about 8 carbon atoms. Examples of such suitable esters nonexclusively include tridecyl neopentanoate, isostearyl palmitate, cetyl ricinoleate, cetyl octanoate, isononyl isononanoate, butyl stearate, octyldodecyl soyate, tridecyl erucate, octyldodecyl erucate/eicosil erucate, and mixtures thereof, with cetyl octanoate, isostearyl palmitate, isononyl isononanoate, and mixtures thereof and being preferred.

Another suitable ester includes the branched or unsaturated C₅ to C₂₂ alkyl alcohol esters of an acid selected from the group consisting of adipic acid, succinic acid, maleic acid, sebacic acid, and mixtures thereof. The alcohol of these esters, which has from about 3 carbon atoms to about 18 carbon atoms, and preferably from about 3 carbon atoms to about 8 carbon atoms, is preferably branched or unsaturated. Examples of such suitable alcohol esters nonexclusively include diisopropyl adipate, dioctyl sebacate, dioctyl succinate, dioctyl maleate, diisostearyl adipate, diethyl sebacate, and mixtures thereof, with diethyl sebacate, dioctyl sebacate, and diisostearyl adipate being preferred.

Another suitable ester includes polyether interrupted fatty acid esters. Examples of such suitable esters nonexclusively include: 1) laureth-2 benzoate; 2) C₈ to C₂₂ fatty alkyl (optionally polypropylenoxy) polyethyleneoxy carboxylate esters derived from an alcohol having from about 1 carbon atom to about 22 carbon atoms, is either straight or branched, and may contain a phenyl group; and 3) mixtures thereof, with C₈ to C₂₂ fatty alkyl (optionally polypropylenoxy) polyethyleneoxy carboxylate esters being preferred. Specific examples of preferred esters nonexclusively include isopropyl propylene glycol-2-isodeceth-7 carboxylate, such as “Velsan D8P3” and other commercially available materials sold by Sandoz under the tradename, “Velsan.”

Another suitable ester includes the benzoic acid esters of heterogeneous alcohols having from about 8 carbon atoms to about 22 carbon atoms, such as the ester mixtures available from Finetex under the tradename, “Finsolv” and preferably is the C₁₂ to C₁₅ alcohols benzoate available from Finetex under the tradename, “Finsolv TN.”

Preferred combinations of esters include at least one, preferably at least two, and more preferably three of the following esters: a) branched C₅ to C₂₂ alkyl alcohol esters of an aromatic acid; b) branched or straight-chained C₅ to C₂₂ alkyl acid esters of branched or unsaturated alkyl alcohols; and c) straight-chained or branched C₅ to C₂₂ alkyl acid esters of optionally ethyoxylated/propoxylated polyols. In a preferred embodiment, the ester contains, based upon the total weight percent of the esters, from about 30 percent to about 80 percent of branched or straight-chained C₅to C₂₂ alkyl acid esters of branched or unsaturated C₅ to C₂₂ alkyl alcohols; from about 10 percent to about 50 percent of branched C₅ to C₂₂ alkyl alcohol esters of an aromatic acid; and from about 10 percent to about 50 percent of straight-chained or branched C₅ to C₂₂ alkyl acid esters of optionally ethyoxylated/propoxylated polyols. In a more preferred embodiment, the ester contains, based upon the total weight percent of the esters, from about 15 percent to about 50 percent isononyl isononanoate, from about 15 percent to about 50 percent isostearyl palmitate, from about 15 percent to about 50 percent cetyl octanoate, and from about 15 percent to about 50 percent pentaerthritol tetraoctanoate.

The cleansing composition of the present invention may further include a volatile or nonvolatile liquid silicone, with the former being preferred. Examples of suitable silicones nonexclusively include the polydimethyl siloxanes and derivatives thereof such as hexamethylsiloxane, dimethicone, dimethiconol, and cyclomethicone, with cyclomethicone being preferred. Examples of suitable cyclomethicones nonexclusively include cyclotetradimethyl siloxane; cyclopentadimethyl siloxane, cyclohexadimethyl siloxane, cycloheptadimethyl siloxane, and mixtures thereof. Preferably, the silicone has a viscosity of from about 0.25 cp to about 350 cp.

Another embodiment of the present invention is directed to a cleansing system comprising, consisting, or consisting essentially of, based upon the total weight of the cleansing system, a) at least 5 percent and preferably at least about 10 percent of the cleansing composition; b) from about 70 percent to about 98 percent, and preferably from about 80 percent to about 90 percent of water; c) from about 0.1 percent to about 5 percent, e.g. from about 0.5 percent to about 1.5 percent of a polymeric emulsifier, a thickener, or mixture thereof; optionally d) from about 0.1 percent to about 5 percent, and preferably from about 1 percent to about 3 percent of a cleansing enhancer; optionally e) from about 2 percent to about 20 percent, and preferably from about 5 percent to about 15 percent of a foaming surfactant; and optionally f) from about 0.001 percent to about 5 percent of a benefit agent.

The cleansing system may be in the form of an oil-in-water emulsion, a water-in-oil emulsion, or a dispersion.

In addition to the cleansing composition, the cleansing system may further be comprised of polymeric emulsifiers and/or thickeners. As used herein, the term “polymeric emulsifier” shall mean those compounds capable of emulsifying cleaning systems whereby the polymeric emulsifiers have a molecular weight of at least about 5000, and preferably are block copolymers having a hydrophilic portion and a hydrophobic portion. When used at amounts effective for emulsifying the cleansing system, the polymeric emulsifiers surprisingly do not cause significant eye sting, i.e., when the emulsifer-containing composition was used by 80 consumers in the eye area, no more than about 5% of such users expressed discomfort around the eye area. Examples of suitable polymeric emulsifiers nonexclusively include polyethylene glycol-30 dipolyhydroxystearate available from Uniqema under the tradename, “Arlacel P-135;” dimethicone copolyol, which is available from Goldschmidt Chemical Corporation under the tradename, “Abil EM 90”; substituted acrylates such as those available from The Goodrich Corporation under the tradename, “Pemulen”; and mixtures thereof, with polyethylene glycol-30 dipolyhydroxystearate being preferred.

Examples of suitable hydrophilic thickeners nonexclusively include carbomers available from B.F. Goodrich under the tradename, “Carbopol ETD 2020”, acrylate copolymers, hydroxyethylcellulose modified with cetyl ether groups available from Hercules under the tradename, “Natrosol Plus”, polyvinylmethyl ether/maleic anhydride (PVM/MA) decadiene crosspolymer available from International Specialty Products under the tradename, “Stabileze QM,” and copolymers and mixtures thereof, with carbomers being preferred. Examples of suitable acrylate copolymers nonexclusively include acrylate copolymers available from Rohm & Haas under the tradename, “Aculyn 33,” acrylates/aminoacrylates copolymer available from National Starch & Chemical Company under the tradename, “Structure Plus,” acrylates/steareth-20 itaconate copolymer available from National Starch & Chemical Company under the tradename, “Structure 2001,” acrylates/ceteth-20 itaconate copolymer available from National Starch & Chemical Company under the tradename, “Structure 3001,” acrylates/steareth-20 methacrylate copolymer available from Rohm & Haas under the tradename, “Aculyn 22,” and copolymers and mixtures thereof.

The cleansing system of the present invention may also optionally contain a cleansing enhancer in the form of a nonionic emulsifier and/or a non-foaming surfactant. Examples of suitable nonionic emulsifiers include isocetheth-20, oleth-2, mixture of PEG-40 hydrogenated castor oil and trideceth-9 available from Dragoco Inc. under the tradename, “Dragoco Solubilizer 2/014160,” Poloxamer 184, laureth4, sorbitan trioleate, polyoxyethylene-(2) oleyl ether, sorbitan stearate, cetearyl glucoside, glyceryl oleate, trideceth-9, polyethylene glycol-40 hydrogenated castor oil, and mixtures thereof.

Examples of suitable non-foaming surfactants include non-foaming nonionic surfactants such as sucrose esters, e.g., sucrose cocoate, sucrose stearate and mixtures thereof, with sucrose cocoate being preferred. By “essentially non-foaming,” it is meant that the surfactant, when used with the composition of the present invention, has a column height of less than about 20 mm as determined by the Ross-Miles Foam Generation Test. See 18 (I.) Oil & Soap 99 - 102 (1941)[“Ross-Miles Test”), which is incorporated by reference herein. The cleansing composition and the cleansing system may either be rinseable with water or may be wiped-off. Preferably, the essentially, non-foaming surfactants are used in embodiments wherein the cleansing system or the cleansing composition is rinseable with water. For example, a preferred combination of hydrophilic components include, based upon the total weight percent of the cleansing system, from about 0.1 percent to about 5.0 percent of hexylene glycol, from about 0.5 percent to about 3.0 percent of sucrose cocoate non foaming surfactant, and from about 0.5 percent to about 3.0 percent of polypropylene glycol stearyl ether. An example of a suitable cleansing enhancer is a mixture of sorbitan stearate and sucrose cocoate available from Uniqema under the tradename, “Arlatone 2121.”

Preferably, the cleansing system contains, based upon the total weight of the cleansing system, no more than about 6%, and preferably 5%, of the cleaning enhancers for cream formulations and no more than about 2%, and preferably no more than 1% of the cleansing enhancers in thin lotion/milk formulations.

The cleansing system and cleansing composition may contain a foaming surfactant. The foaming surfactant may be non-ionic, cationic, amphoteric, or anionic; nonionic surfactants are preferred. By “foaming,” it is meant that the surfactant, when used with the composition of the present invention, has a column height of foam greater than about 20 mm as determined by the Ross-Miles Test. As used herein, the term “amphoteric” shall mean: 1) molecules that contain both acidic and basic sites such as, for example, an amino acid containing both amino (basic) and acid (e.g., carboxylic acid, acidic) functional groups; or 2) zwitterionic molecules which possess both positive and negative charges within the same molecule. The charges of the latter may be either dependent on or independent of the pH of the composition. Examples of zwitterionic materials include, but are not limited to, alkyl betaines and amidoalkyl betaines. Examples of suitable and preferred surfactants may be found in International Patent Application Number WO97/01196, which is incorporated by reference in its entirety herein.

The cleansing system may further contain one or more benefit agents or pharmaceutically-acceptable salts thereof. As used herein, the term “benefit agent” includes any active ingredient that is to be delivered into and/or onto the skin, hair or nail at a desired location, such as a cosmetic agent or a pharmaceutical agent. By “cosmetic agent,” it is meant any ingredient that is appropriate for cosmetically treating, providing nutrients to, and/or conditioning the hair, nail, and/or skin via topical application. By “pharmaceutical agent,” it is mean any drug that is either hydrophobic or hydrophilic in nature and appropriate for topical use. As used herein “medicament agents” include those agents capable of promoting recovery from injury and illness.

The benefit agents useful herein may be categorized by their therapeutic benefit or their postulated mode of action. However, it is to be understood that the benefit agents useful herein may, in some circumstances, provide more than one thereapeutic benefit or operate via greater than one mode of action. Therefore, the particular classifications provided herein are made for the sake of convenience and are not intended to limit the benefit agents to the particular application(s) listed. In addition, the compounds, which are identified below as being suitable for use as benefit agents, may be used in an amount over and above the amount that they may be used for other purposes in the cleansing composition/cleansing system.

Examples of suitable benefit agents include, but are not limited to, depigmentation agents; reflectants; detangling/wet combing agents; film forming polymers; humectants; amino acids and their derivatives; antimicrobial agents; allergy inhibitors; anti-acne agents; anti-aging agents; anti-wrinkling agents, antiseptics; analgesics; antitussives; antipruritics; local anesthetics; anti-hair loss agents; hair growth promoting agents; hair growth inhibitor agents, antihistamines such as Mandragora Vernalis, Tanacetum Parthenium and the like; antiinfectives such as Acacia Catechu, Aloe Barbadensis, Convallaria Majalis, Echinacea, Eucalyptus, Mentha Piperita, Rosa Canina, Sassafras Albidum, and the like; inflammation inhibitors; anti-emetics; anticholinergics; vasoconstrictors; vasodilators; wound healing promoters; peptides, polypeptides and proteins; deodorants and anti-perspirants; medicament agents; skin emollients and skin moisturizers; skin firming agents, hair conditioners; hair softeners; hair moisturizers; vitamins; tanning agents; skin lightening agents; antifungals such as Centaurea Cyanus, Kalmia Latifolia and antifungals for foot preparations; depilating agents; shaving preparations; external analgesics; perfumes; counterirritants; hemorrhoidals; insecticides; poison ivy products; poison oak products; burn products; anti-diaper rash agents; prickly heat agents; make-up preparations; vitamins; amino acids and their derivatives; herbal extracts; retinoids; flavenoids; sensates; anti-oxidants; skin conditioners; hair lighteners; chelating agents; cell turnover enhancers; coloring agents; pigments; sunscreens, those active ingredients disclosed in U.S. Pat. No. 6,063,397, which is incorporated herein by reference, anti-edema agents, collagen enhancers, and mixtures thereof.

Examples of suitable anti-edema agents nonexclusively include bisabolol natural, synthetic bisabolol, and mixtures thereof.

Examples of suitable vasoconstrictors nonexclusively include horse chestnut extract, prickly ash, and mixtures thereof.

Examples of suitable anti-inflammatory agents nonexclusively include benoxaprofen, centella asiatica, bisabolol, feverfew (whole), feverfew (parthenolide free), green tea extract, green tea concentrate, hydrogen peroxide, lycopene including “Lyc-o-Pen” available from LycoRed Natural Products Industries, Ltd., oat oil, chamomile, and mixtures thereof.

Examples of collagen enhancers nonexclusively include vitamin A, vitamin C, and mixtures thereof.

Examples of suitable skin firming agent nonexclusively include dimethylaminoethanol (“DMAE”).

Examples of suitable antipruritics and skin protectants nonexclusively include oatmeal, betaglucan, feverfew, soy and derivatives thereof, bicarbonate of soda, colloidal oatmeal, surfactant based colloidal oatmeal cleanser, Anagallis Arvensis, Oenothera Biennis, Verbena Officinalis, and the like. These antipruritics may be used in an amount, based upon the total weight of the cleansing composition, from about 0.01 percent to about 40 percent, and preferably from about 1 percent to about 5 percent.

As used herein, colloidal oatmeal means the powder resulting from the grinding and further processing of whole oat grain meeting United States Standards for Number 1 or Number 2 oats. The colloidal oatmeal has a particle size distribution as follows: not more than 3 percent of the total particles exceed 150 micrometers in size and not more than 20 percent of the total particles exceed 75 micrometers in size. Examples of suitable colloidal oatmeals include, but are not limited to, “Tech-0” available from the Beacon Corporation and colloidal oatmeals available from Quaker.

Examples of suitable reflectants nonexclusively include mica, alumina, calcium silicate, glycol dioleate, glycol distearate, silica, sodium magnesium fluorosilicate, and mixtures thereof.

Suitable detangling/wet combing agents nonexclusively include polyquaternium-10, hydroxypropyltrimonium guar, dioleoylamidoethyl hydroxyethylmonium methosulfate, di-(soyoylethyl) hydroxyethylmonium methosulfate, hydroxyethyl behenamidopropyl dimonium chloride, olealkonium chloride, polyquaternium-47, stearalkonium chloride, tricetylmonium chloride, and mixtures thereof.

Suitable film forming polymers include those that, upon drying, produce a substantially continuous coating or film on the hair, skin, or nails. Nonexclusive examples of suitable film forming polymers include acrylamidopropyl trimonium chloride/acrylamide copolymer; corn starch/ acrylamide/sodium acrylate copolymer; polyquaternium-10; polyquaternium-47; polyvinylmethylether/maleic anhydride copolymer; styrene/acrylates copolymers; and mixtures thereof.

Commercially available humectants which are capable of providing moisturization and conditioning properties to the cleansing composition are suitable for use in the present invention. The humectant is preferably present in an amount of from about 0 percent to about 10 percent, more preferably from about 0.5 percent to about 5 percent, and most preferably from about 0.5 percent to about 3 percent, based on the overall weight of the composition. Examples of suitable humectants nonexclusively include: 1) water soluble liquid polyols selected from the group comprising glycerine, propylene glycol, hexylene glycol, butylene glycol, pentylene glycol, dipropylene glycol, and mixtures thereof; 2) polyalkylene glycol of the formula I.:
HO—(R″O)_{b}—H I.
wherein R″ is an alkylene group having from about 2 to about 4 carbon atoms and b is an integer of from about 1 to about 10, such as PEG 4; 3) polyethylene glycol ether of methyl glucose of formula II.:
CH₃—C₆H₁₀O₅—(OCH₂CH₂)_{c}—OH II.
wherein c is an integer from about 5 to about 25;
4) urea; 5) fructose; 6) glucose; 7) honey; 8) lactic acid; 9) maltose; 10) sodium glucuronate; and 11) mixtures thereof, with glycerine being the preferred humectant.

Suitable amino acid agents include amino acids derived from the hydrolysis of various proteins as well as the salts, esters, and acyl derivatives thereof. Examples of such amino acid agents nonexclusively include amphoteric amino acids such as alkylamido alkylamines, i.e. stearyl acetyl glutamate, capryloyl silk amino acid, capryloyl collagen amino acids; capryloyl keratin amino acids; capryloyl pea amino acids; cocodimonium hydroxypropyl silk amino acids; corn gluten amino acids; cysteine; glutamic acid; glycine; hair keratin amino acids; amino acids such as aspartic acid, threonine, serine, glutamic acid, proline, glycine, alanine, cystine, valine, methionine, isoleucine, leucine, tyrosine, phenylalanine, cysteic acid, lysine, histidine, arginine, cysteine, tryptophan, citrulline; lysine; silk amino acids, wheat amino acids; and mixtures thereof

Suitable proteins include those polymers that have a long chain, i.e. at least about 10 carbon atoms, and a high molecular weight, i.e. at least about 1000, and are formed by self-condensation of amino acids. Nonexclusive examples of such proteins include collagen, deoxyribonuclease, iodized corn protein; milk protein; protease; serum protein; silk; sweet almond protein; wheat germ protein; wheat protein; alpha and beta helix of keratin proteins; hair proteins, such as intermediate filament proteins, high-sulfur proteins, ultrahigh-sulfur proteins, intermediate filament-associated proteins, high-tyrosine proteins, high-glycine tyrosine proteins, tricohyalin, and mixtures thereof.

Examples of suitable vitamins nonexclusively include vitamin B complex; including thiamine, nicotinic acid, biotin, pantothenic acid, choline, riboflavin, vitamin B6, vitamin B12, pyridoxine, inositol, carnitine; vitamins A,C,D,E,K and their derivatives such as vitamin A palmitate and pro-vitamins, e.g. (i.e. panthenol (pro vitamin B5) and panthenol triacetate) and mixtures thereof.

Examples of suitable antibacterial agents nonexclusively include bacitracin, erythromycin, neomycin, tetracycline, chlortetracycline, benzethonium chloride, phenol, and mixtures thereof.

Examples of suitable skin emollients and skin moisturizers nonexclusively include mineral oil, lanolin, vegetable oils, isostearyl isostearate, glyceryl laurate, methyl gluceth-10, methyl gluceth-20 chitosan, and mixtures thereof.

Examples of suitable hair conditioners nonexclusively include quaternized compounds such as behenamidopropyl PG-dimonium chloride, tricetylmonium chloride, dihydrogenated tallowamidoethyl hydroxyethylmonium methosulfate, and mixtures thereof as well as lipophilic compounds like cetyl alcohol, stearyl alcohol, hydrogenated polydecene, and mixtures thereof.

An example of a suitable hair softener nonexclusively includes silicone compounds, such as those that are either non-volatile or volatile and those that are water soluble or water insoluble. Examples of suitable silicones include organo-substituted polysiloxanes, which are either linear or cyclic polymers of monomeric silicone/oxygen monomers and which nonexclusively include cetyl dimethicone; cetyl triethylammonium dimethicone copolyol phthalate; cyclomethicone; dimethicone copolyol; dimethicone copolyol lactate; hydrolyzed soy protein/dimethicone copolyol acetate; silicone quaternium 13; stearalkonium dimethicone copolyol phthalate; stearamidopropyl dimethicone; and mixtures thereof.

Examples of suitable hair moisturizers nonexclusively include panthenyl ethyl ether, phytantriol, and mixtures thereof.

Examples of sunscreen agents nonexclusively include benzophenones, bornelone, butyl paba, cinnamidopropyl trimethyl ammonium chloride, disodium distyrylbiphenyl disulfonate, paba, potassium methoxycinnamate, butyl methoxydibenzoylmethane, octyl methoxycinnamate, oxybenzone, octocrylene, octyl salicylate, phenylbenzimidazole sulfonic acid, ethyl hydroxypropyl aminobenzoate, menthyl anthranilate, aminobenzoic acid, cinoxate, diethanolamine methoxycinnamate, glyceryl aminobenzoate, titanium dioxide, zinc oxide, oxybenzone, Padimate O, red petrolatum, and mixtures thereof.

An example of a suitable tanning agent nonexclusively includes dihydroxyacetone.

Examples of skin lightening agents nonexclusively include hydroquinone, catechol and its derivatives, ascorbic acid and its derivatives, and mixtures thereof.

Examples of suitable insecticides (including insect repellents, anti-scabies and anti-lice treatments) nonexclusively include permethrin, pyrethrin, piperonyl butoxide, imidacloprid, N,N-diethyl toluamide, which refers to the material containing predominantly the meta isomer, i.e., N,N-diethyl-m-toluamide, which is also known as DEET; natural or synthetic pyrethroids, whereby the natural pyrethroids are contained in pyrethrum, the extract of the ground flowers of Chrysanthemum cinerariaefolium or C coccineum; and mixtures thereof. Ethyl 3-(N-butylacetamido)propionate, which is available commercially from Merck KGaA of Darmstadt, Germany under the name, “Insect Repellent 3535” is also useful.

An example of an anti fungal for foot preparations nonexclusively includes tolnaftate.

Examples of suitable depilating agents nonexclusively include calcium thioglycolate, magnesium thioglycolate, potassium thioglycolate, strontium thioglycolate, and mixtures thereof.

Examples of suitable external analgesics and local anesthetics nonexclusively include benzocaine, dibucaine, benzyl alcohol, camphor, capsaicin, capsicum, capsicum oleoresin, juniper tar, menthol, methyl nicotinate, methyl salicylate, phenol, resorcinol, turpentine oil, and mixtures thereof.

Examples of suitable antiperspirants and deodorants nonexclusively include aluminium chlorohydrates, aluminium zirconium chlorohydrates, and mixtures thereof.

Examples of suitable counterirritants nonexclusively include camphor, menthol, methyl salicylate, peppermint and clove oils, ichtammol, and mixtures thereof.

An example of a suitable inflammation inhibitor nonexclusively includes hydrocortisone, Fragaria Vesca, Matricaria Chamomilla, and Salvia Officinalis.

Examples of suitable hemorrhoidal products nonexclusively include the anesthetics such as benzocaine, pramoxine hydrochloride, and mixtures thereof; antiseptics such as benzethonium chloride; astringents such as zinc oxide, bismuth subgallate, balsam Peru, and mixtures thereof; skin protectants such as cod liver oil, vegetable oil, and mixtures thereof.

Most preferred benefit agents nonexclusively include DMAE, soy and derivatives thereof, colloidal oatmeal, sulfonated shale oil, olive leaf, elubiol, 6-(1 -piperidinyl)-2,4-pyrimidinediamine-3-oxide, finasteride, ketoconazole, salicylic acid, zinc pyrithione, coal tar, benzoyl peroxide, selenium sulfide, hydrocortisone, sulfur, menthol, pramoxine hydrochloride, tricetylmonium chloride, polyquaternium 10, panthenol, panthenol triacetate, vitamin A and derivatives thereof, vitamin B and derivatives thereof, vitamin C and derivatives thereof, vitamin D and derivatives thereof, vitamin E and derivatives thereof, vitamin K and derivatives thereof, keratin, lysine, arginine, hydrolyzed wheat proteins, hydrolyzed silk proteins, octyl methoxycinnamate, oxybenzone, minoxidil, titanium dioxide, zinc dioxide, retinol, erthromycin, tretinoin, and mixtures thereof.

One preferred type of benefit agent includes those therapeutic components that are effective in the treatment of dandruff, seborrheic dermatitis, and psoriasis as well as the symptoms associated therewith. Examples of such suitable benefits agents nonexclusively include zinc pyrithione, anthralin, shale oil and derivatives thereof such as sulfonated shale oil, selenium sulfide, sulfur; salicylic acid; coal tar; povidone-iodine, imidazoles such as ketoconazole, dichlorophenyl imidazolodioxalan, which is commercially available from Janssen Pharmaceutica, N.V., under the tradename, “Elubiol”, clotrimazole, itraconazole, miconazole, climbazole, tioconazole, sulconazole, butoconazole, fluconazole, miconazole nitrate and any possible stereo isomers and derivatives thereof; piroctone olamine (Octopirox); selenium sulfide; ciclopirox olamine; anti-psoriasis agents such as vitamin D analogs, e.g. calcipotriol, calcitriol, and tacaleitrol; vitamin A analogs such as esters of vitamin A, e.g. vitamin A palmitate, retinoids, retinols, and retinoic acid; corticosteroids such as hydrocortisone, clobetasone, butyrate, clobetasol propionate and mixtures thereof.

The amount of benefit agent to be combined with the cleansing composition or the emulsion may vary depending upon, for example, the ability of the benefit agent to penetrate through the skin, hair or nail, the specific benefit agent chosen, the particular benefit desired, the sensitivity of the user to the benefit agent, the health condition, age, and skin, hair, and/or nail condition of the user, and the like. In sum, the benefit agent is used in a “safe and effective amount,” which is an amount that is high enough to deliver a desired skin, hair or nail benefit or to modify a certain condition to be treated, but is low enough to avoid serious side effects, at a reasonable risk to benefit ratio within the scope of sound medical judgment. Unless otherwise expressed herein, typically the benefit agent is present in the cleansing system in an amount, based upon the total weight of the system, from about 0.01 percent to about 5.0 percent, and preferably from about 0.01 percent to about 2.0 percent, and more preferably from about 0.01 percent to about 1.0 percent.

Optionally, commercially available detergent thickeners that are capable of imparting the appropriate viscosity to conditioning shampoo compositions are suitable for use in this invention. If used, the detergent thickeners should be present in the shampoo compositions in an amount sufficient to raise the Brookfield viscosity of the composition to a value of between about 500 to about 10,000 centipoise. Examples of suitable detergent thickeners nonexclusively include: mono or diesters of polyethylene glycol of formula IV.
HO—(CH₂CH₂O)_{z}H IV.
wherein z is an integer from about 3 to about 200;
fatty acids containing from about 16 to about 22 carbon atoms; fatty acid esters of ethoxylated polyols; ethoxylated derivatives of mono and diesters of fatty acids and glycerine; hydroxyalkyl cellulose; alkyl cellulose; hydroxyalkyl alkyl cellulose; and mixtures thereof. More specifically, suitable detergent thickeners nonexclusively include behenalkonium chloride; cetyl alcohol, quaternium-46, hydroxyethyl cellulose, cocodimonium chloride, polyquaternium-6, polyquaternium-7, quaternium-18, PEG-18 glycerol oleate/cocoate, a mixture of acrylates/steareth-50 acrylate copolymer, laureth-3 and propylene glycol, which is commercially available from Goldschmidt under the tradename “Antil 208,” a mixture of cocamidopropylbetaine and glyceryl laurate which is commercially available from Goldschmidt under the tradename, “Antil HS60,” a mixture of propylene glycol, PEG 55, and propylene glycol oleate, which is commercially available from Goldschmidt under the tradename, “Antil 414 liquid,” and mixtures thereof. Preferred detergent thickeners include polyethylene glycol ester, and more preferably PEG-150 distearate which is available from the Stepan Company of Northfield, Ill. or from Comiel, S.p.A. of Bologna, Italy under the tradename, “PEG 6000 DS”.

The above described cleansing composition and cleansing system may be prepared by combining the desired components in a suitable container and mixing them under ambient conditions in any conventional mixing means well known in the art, such as a mechanically stirred propeller, paddle, and the like.

In another preferred embodiment of the cleansing system of the present invention wherein a polymeric emulsifier such as, for example, polyethylene glycol-30 dipolyhydroxystearate (hereinafter “PEG 30”) or dimethicone copolyol, are used and water is used as the vehicle, an oil-in-water emulsion may be produced. Although both the PEG 30 and dimethicone copolyol are marketed for use in formulating water-in-oil compositions, we have unexpectedly found that oil-in-water emulsions may be created due to the unique processing steps and conditions employed herein. More specifically, we found that when a thickening agent, preferably a hydrophilic thickening agent, is neutralized in the hydrophilic phase of the present invention comprising a polymeric emulsifier prior to adding the lipophilic phase of the present invention thereto, the resulting emulsion is in the form of a water-in-oil emulsion. Conversely, when a thickening agent, preferably a hydrophilic thickening agent, is neutralized in the hydrophilic phase of the present invention comprising a polymeric emulsifier after the lipophilic phase of the present invention is added to the hydrophilic phase, the resulting emulsion is unexpectedly in the form of a oil-in-water emulsion.

Cleansing systems of the present invention that are emulsions may contain, based upon the total weight of the emulsion, from about 0.01 percent to about 2 percent, and preferably from about 0.01 percent to about 0.5 percent of hydrophilic thickeners. Suitable neutralizers include any known bases, such as sodium hydroxide, or acids, such as lactic acid, that are capable of neutralizing the hydrophilic thickening agent, in either the hydrophilic phase (if a water-in-oil emulsion is desired) or a mixture of both the hydrophilic phase and the lipophilic phase (if an oil-in-water emulsion is desired) of the present invention to a pH of about 5 to about 7 under ambient temperature. In one embodiment, hydrophilic thickeners including acrylates/aminoacrylates copolymer, acrylates/steareth-20 itaconate copolymer, acrylates/ceteth-20 itaconate copolymer, are preferably neutralized with an acid, such as lactic acid. Hydrophilic thickeners including carbomers, modified hydroxyethylcellulose, polyvinylacetate/maleic anhydride (PVA/MA) decadiene crosspolymer, and acrylates/steareth-20 methacrylate copolymer, are preferably neutralized with a base, such as sodium hydroxide (20%).

In one embodiment, the hydrophilic phase may be comprised of one or more of the following components: water, thickener, cleansing enhancer, nonfoaming surfactant, and water dispersible component, and the lipophilic phase may be comprised of one or more of the following components: silicone, ether, and polymeric emulsifier.

Another embodiment of this invention is directed to a foaming composition comprising, based upon the foaming composition, from about 0.1 percent to about 30 percent, e.g. from about 0.1 percent to about 5 percent of a water dispersible component; from about 0.1 percent to about 30 percent, e.g. from about 0.1 percent to about 5 percent of a liquid, water-insoluble propoxylated fatty alcohol; from about 1.0 percent to about 98 percent, e.g. from about 30 percent to about 98 percent or from about 45 percent to about 90 percent of water; and from about 2.0 percent to about 20 percent, e.g. from about 5.0 percent to about 15 percent of a foaming surfactant.

Optionally, the foaming composition may also be comprised of one or more of the following components, based upon the total weight of the foaming composition: a) from about 0.1 percent to 5 percent, e.g. from about 0.5 percent to about 1.5 percent of a polymeric emulsifier, a thickener, or mixture thereof; b) from about 0.1 percent to about 5 percent, e.g. from about 1 percent to about 3 percent of a cleansing enhancer; c) from about 0.001 percent to about 5 percent of a benefit agent; and d) from about 0.1 percent to about 30 percent, e.g. from about 0.1 percent to about 5 percent of a liquid silicone.

Another embodiment of the present invention is directed to a method for depositing a benefit agent onto the skin, hair and/or nails comprised of applying either the above-described cleansing system or cleansing composition with an effective amount of a benefit agent to a desired location on a human or animal. While the frequency and amount of the benefit agent-containing cleaning system to be applied will depend upon, for example, the type and amount of benefit agent available, the intended usage of the final composition, i.e. therapeutic versus maintenance regimen, the amount and type of detergent present, and the sensitivity of the individual user to the composition/emulsion, typically the benefit agent-containing cleaning system of the present invention should be topically applied to affected body parts at regular intervals, and preferably from about 2 to about 14 times per week. More preferably, the composition/emulsion is applied more frequently during the initial stages of treatment, e.g. from about 5 to about 7 times per week until the desired effect is achieved, then less frequently when maintenance is desired, e.g. from about 2 to about 5 times per week.

We have unexpectedly found that the above-described cleansing composition and cleansing system are capable of efficiently mediating the deposition and permeation of various benefit agents, such as antidandruff agents, onto and into the skin following topical administration thereto. More specifically, we have surprisingly found that when benefit agents are combined with either the cleansing composition or the cleaning system of the present invention, the amount of benefit agents deposited onto and/or into the skin, hair, and/or nails is about 50% greater than the amount of benefit agents deposited onto and/or into the skin, hair, and/or nails after application of known, commercial benefit agent-containing cleansers.

An alternative preferred embodiment of the present invention is directed to a method for treating hair loss, such as hair loss resulting from alopecia, comprising topically applying the above-described cleaning system and the hair loss benefit agent to a desired location on an animal or human, wherein the benefit agent is comprised of an effective amount of a hair loss treatment agent such as minoxidil or mixture thereof. As used herein, “hair loss treatment agents” shall include agents capable of growing hair and/or agents capable of preventing the loss of hair. By “effective amount,” it is meant an amount effective for treating hair loss and preferably may range from, based upon the total weight of the cleansing system, from about 0.001 percent to about 20 percent, and preferably from about 1 percent to about 5 percent.

Examples of benefit agents suitable for treating hair loss include, but are not limited to potassium channel openers or peripheral vasodilators such as minoxidil, diazoxide, and compounds such as N*-cyano-N-(tert-pentyl)-N′-3-pyridinyl-guanidine (“P-1075”) as disclosed in U.S. Pat. No.: 5,244,664, which is incorporated herein by reference; vitamins, such as vitamin E and vitamin C, and derivatives thereof such as vitamin E acetate and vitamin C palmitate; hormones, such as erythropoietin, prostaglandins, such as prostaglandin EI and prostaglandin F2-alpha; fatty acids, such as oleic acid; diruretics such as spironolactone; heat shock proteins (“HSP”), such as HSP 27 and HSP 72; calcium channel blockers, such as verapamil HCL, nifedipine, and diltiazemamiloride; immunosuppressant drugs, such as cyclosporin and Fk-506; 5 alpha-reductase inhibitors such as finasteride; growth factors such as, EGF, IGF and FGF; transforming growth factor beta; tumor necrosis factor; non-steroidal anti-inflammatory agents such as benoxaprofen; retinoids such as tretinoin; cytokines, such as IL-6, IL-1 alpha, and IL-1 beta; cell adhesion molecules such as ICAM; glucorcorticoids such as betametasone; botanical extracts such as aloe, clove, ginseng, rehmannia, swertia, sweet orange, zanthoxylum, Serenoa repens (saw palmetto), Hypoxis rooperi, stinging nettle, pumpkin seeds, and rye pollen; other botanical extracts including sandlewood, red beet root, chrysanthemum, rosemary, burdock root and other hair growth promoter activators which are disclosed in DE 4330597 which is incorporated by reference in its entirety herein; homeopathic agents such as Kalium Phosphoricum D2, Azadirachta indica D2, and Joborandi DI; genes for cytokines, growth factors, and male-pattered baldness; antifungals such as ketoconazole and elubiol; antibiotics such as streptomycin; proteins inhibitors such as cycloheximide; acetazolamide; benoxaprofen; cortisone; diltiazem; hexachlorobenzene; hydantoin; nifedipine; penicillamine; phenothaiazines; pinacidil; psoralens, verapamil; zidovudine; alpha-glucosylated rutin having at least one of the following rutins: quercetin, isoquercitrin, hespeddin, naringin, and methylhesperidin, and flavonoids and transglycosidated derivatives thereof which are all disclosed in JP 7002677, which is incorporated by reference in its entirety herein; and mixtures thereof.

Preferred hair loss treatment agents include minoxidil, 6-(1-piperdinyl)-2,4-pyrimidinediamine-3-oxide, N′-cyano-N-(tert-pentyl)-N′-3-pyridinyl-guanidine, finasteride, retinoids and derivatives thereof, ketoconazole, elubiol or mixtures thereof.

Another embodiment of the present invention is directed to a method for inhibiting hair growth comprising topically applying the above-described composition/system combined with a benefit agent to a desired area on an animal or human for inhibiting hair growth, wherein the benefit agent is comprised of an effective amount of a hair growth inhibiting agent. In a preferred embodiment, the cleaning system contains, based upon the total weight of the cleaning system, from about 0.001 percent to about 20 percent, and preferably from about 0.01 percent to about 5 percent hair growth inhibiting agent.

Examples of benefit agents suitable for use in inhibiting hair growth include: serine proteases such as trypsin; vitamins such as alpha-tocophenol (vitamin E) and derivatives thereof such as tocophenol acetate and tocophenol palmitate; antineoplastic agents, such as doxorubicin, cyclophosphamide, chlormethine, methotrexate, fluorouracil, vincristine, daunorubicin, bleomycin and hydroxycarbamide; anticoagulants, such as heparin, heparinoids, coumaerins, detran and indandiones; antithyroid drugs, such as iodine, thiouracils and carbimazole; lithium and lithium carbonate; interferons, such as interferon alpha, interferon alpha-2a and interferon alpha-2b; retinoids, such as retinol (vitamin A), isotretinoin: glucocorticoids such as betamethasone, and dexamethosone; antihyperlipidaemic drugs, such as triparanol and clofibrate; thallium; mercury; albendazole; allopurinol; amiodarone; amphetamines; androgens; bromocriptine; butyrophenones; carbamazepine; cholestyramine; cimetidine; clofibrate; danazol; desipramine; dixyrazine; ethambutol; etionamide; fluoxetine; gentamicin, gold salts; hydantoins; ibuprofen; impramine; immunoglobulins; indandiones; indomethacin; intraconazole; levadopa; maprotiline; methysergide; metoprolol; metyrapone; nadolol; nicotinic acid; potassium thiocyanate; propranolol; pyridostimine; salicylates; sulfasalazine; terfenadine; thiamphenicol; thiouracils; trimethadione; troparanol; valproic acid; and mixtures thereof.

Preferred hair growth inhibitory agents include serene proteases, retinol, isotretinoin, betamethoisone, alpha-tocophenol and derivatives thereof, or mixtures thereof.

Another preferred embodiment of the present invention is directed to a method for treating acne and for reducing the signs of aging, i.e. wrinkles, fine lines, and other manifestations of photodamage, comprising topically applying the above-described cleaning system and the relevant benefit agent to the skin of an animal or human at a desired area, wherein the benefit agent is comprised of an effective amount of an anti-acne agent or an anti-aging agent, respectively.

Examples of suitable anti-aging agents include, but are not limited to inorganic sunscreens such as titanium dioxide and zinc oxide; organic sunscreens such as octyl-methoxy cinnamates and derivatives thereof; retinoids; vitamins such as vitamin E, vitamin A, vitamin C, vitamin B, and derivatives thereof such as vitamin E acetate, vitamin C palmitate, and the like; antioxidants including beta carotene, alpha hydroxy acids such as glycolic acid, citric acid, lactic acid, malic acid, mandelic acid, ascorbic acid, alpha-hydroxybutyric acid, alpha-hydroxyisobutyric acid, alpha-hydroxyisocaproic acid, atrrolactic acid, alpha-hydroxyisovaleric acid, ethyl pyruvate, galacturonic acid, glucoheptonic acid, glucoheptono 1,4-lactone, gluconic acid, gluconolactone, glucuronic acid, glucuronolactone, glycolic acid, isopropyl pyruvate, methyl pyruvate, mucic acid, pyruvic acid, saccharic acid, saccaric acid 1,4-lactone, tartaric acid, and tartronic acid; beta hydroxy acids such as beta-hydroxybutyric acid, beta-phenyl-lactic acid, beta-phenylpyruvic acid; botanical extracts such as green tea, soy, milk thistle, algae, aloe, angelica, bitter orange, coffee, goldthread, grapefruit, hoellen, honeysuckle, Job's tears, lithospermum, mulberry, peony, puerarua, nice, safflower, and mixtures thereof.

Preferred anti-aging agents include retinoids, anti-oxidants, alpha-hydroxy acids and beta-hydroxy acid with retinol and tretinoin being most preferred.

Suitable amounts of anti-aging agents include, based upon the total weight of the described cleaning system, from about 0.01 percent to about 10 percent, and preferably from about 0.04 percent to about 5 percent.

Examples of suitable anti-acne agents include, but are not limited to topical retinoids (tretinoin, isotretinoin, motretinide, adapalene, tazarotene, azelaic acid, retinol); salicylic acid; benzoyl peroxide; resorcinol; antibiotics such as tetracycline and isomers thereof, erythromycin, and the anti-inflammatory agents such as ibuprofen, naproxen, hetprofen; botanical extracts such as alnus, arnica, artemisia capillaris, asiasarum root, birrh, calendula, chamomile, cnidium, comfrey, fennel, galla rhois, hawthorn, houftuynia, hypericum, jujube, kiwi, licorice, magnolia, olive, peppermint, philodendron, salvia, sasa albo-marginata; imidazoles such as ketoconazole and elubiol, and those described in Gollnick, H et al. 196(I) Dermatology Sebaceous Glands, Acne and Related Disorders, 119-157 (1998), which is incorporated by reference herein, and mixtures thereof.

Preferred anti-acne agents include benzoyl peroxide, retinol, elubiol, antibiotics, and salicylic acid, with retinol and tretinoin being most preferred.

Suitable amounts of anti-acne agents include, based upon the total weight of the described cleansing system, from about 0.01 percent to about 10 percent, and preferably from about 0.04 percent to about 5 percent.

Another preferred embodiment of the present invention is directed to a method for depigmenting the skin, comprising topically applying to skin at a desired area the above-described cleaning system and an effective amount of the depigmentation benefit agent. Suitable effective amounts of depigmentation agents include, based upon the total weight of the described cleaning system, from about 0.01 percent to about 10 percent, and preferably from about 0.04 percent to about 5 percent.

Examples of suitable depigmentation agents include, but are not limited to soy and derivatives thereof, retinoids such as retinol; Kojic acid and its derivatives such as, for example, kojic dipalmitate; hydroquinone and it derivatives such as arbutin; transexamic acid; vitamins such as niacin, vitamin C and its derivatives; azelaic acid; placertia; licorice; extracts such as chamomile and green tea, and mixtures thereof, with retinol, Kojic acid, and hydroquinone, being preferred.

An alternative preferred embodiment of the present invention is directed to a method for treating the symptoms and/or the diseases of dandruff, seborrheic dermatitis and/or psoriasis, comprising topically applying the above-described cleaning system and the relevant benefit agent to a location desired wherein the benefit agent is comprised of an effective amount of a dandruff treatment agent, a seborrheic dermatitis treatment agent, or a psoriasis treatment agent, respectively. As used herein, “dandruff treatment agent,” “seborrheic dermatitis treatment agent,” or a “psoriasis treatment agent,” respectively, shall include agents capable of treating the symptoms and/or the diseases of dandruff, seborrheic dermatitis, and psoriasis, respectively. By “effective amount,” it is meant an amount effective for treating the disease and/or the symptoms associated therewith and preferably may range from, based upon the total weight of the cleaning system, from about 0.001 percent to about 10 percent, and preferably from about 0.01 percent to about 5 percent.

Examples of benefit agents suitable for treating the symptoms and/or the diseases of dandruff, seborrheic dermatitis and/or psoriasis, respectively, nonexclusively include those set forth above with shale oil and derivatives thereof, elubiol, ketoconazole, coal tar, salicylic acid, zinc pyrithione, selenium sulfide, hydrocortisone, sulfur, menthol, pramoxine hydrochloride, and mixtures thereof being particularly preferred.

The compositions of the present invention may be directed applied to the skin or may be applied onto other delivery implements such as wipes, sponges, brushes, and the like. The compositions may be used in products designed to be left on the skin, wiped from the skin, or rinsed off of the skin.

Several examples are set forth below to further illustrate the nature of the invention and the manner of carrying it out. However, the invention should not be considered as being limited to the details thereof.

### EXAMPLE 1

### Fragrance Free Foaming Facial Cleanser

The materials listed in Table 1 were combined as described below to generate a fragrance free foaming facial cleanser.

TABLE 1

| | | | Weight |
|---|---|---|---|
| Trade Name | Function | INCI Name | % |
| Water | Vehicle | Water | 69.8 |
| Disodium EDTA | Chelating agent | Disodium EDTA | 0.1 |
| Glycerin 917 | Humectant | Glycerin 99.7% | 3.0 |
| RHODICARE T | Polymer | Xanthan Gum | 0.1 |
| MONATERIC | Surfactant | Disodium | 8.0 |
| 949J | | Lauroamphodiacetate | |
| TEGOBETAINE | Surfactant | Cocamidopropyl Betaine | 7.0 |
| L7 | | | |
| RHODACAL A- | Surfactant | Sodium C₁₄-C₁₆ Olefin | 6.0 |
| 246/L | | Sulphonate | |
| GLYCEROX | Emollient | PEG-6 Caprylic/Capric | 1.0 |
| 767 | | Glyceride | |
| ARLAMOL E | Emulsifier | PPG-15 Stearyl Ether | 1.0 |
| Hexylene Glycol | Solvent | Hexylene Glycol | 1.0 |
| PHENONIP | Preservative | Phenoxyethanol + Parabens | 1.0 |
| TWEEN 20 | Viscosity | Polysorbate 20 | 2.0 |
| | adjuster | | |
| AQUAFLEUR | Fragrance | Aqua Fleur | 0 |
| Mod A | | | |
| Citric Acid | PH adjuster | Citric Acid (20% solution) | 8.0 |

To a main batch vessel was added the water and Disodium EDTA. The vessel was heated to 40-45° C. The vessel was stirred until the EDTA dissolved. In a separate vessel, the RHODICARE T and the Glycerin 917 were mixed until the RHODICARE dissolved. The RHODICARE/Glycerin mixture was then added to the main batch and stirred for 30 minutes. The MONATERIC 949J, the TEGOBETAINE L7, and the RHODACAL A-246/L were then added to the main batch and stirred until dissolved. The batch was cooled. The GLYCEROX 767, ARLAMOL E, and Hexylene Glycol were added during cooling and stirred until dispersed. The PHENONIP and the TWEEN 20 was then added to the main batch and stirred until dispersed. The pH was measured and adjusted to 6.1 using the citric acid solution. The product was clear. Sub-samples were taken and stored at 4° C., 40° C., and 50° C. The samples were checked over time to see if they were physically stable (did not phase separate). None of the samples phase separated after one month storage at the specified temperatures.

### EXAMPLE 2

### Fragranced Foaming Facial Cleanser

The materials listed in Table 2 were combined as described below to generate a fragranced foaming facial cleanser.

TABLE 2

| Trade Name | Function | INCI Name | Weight % |
|---|---|---|---|
| Water | Vehicle | Water | 68.9 |
| Disodium EDTA | Chelating | Disodium EDTA | 0.1 |
| | agent | | |
| Glycerin 917 | Humectant | Glycerin 99.7% | 3.0 |
| RHODICARE T | Polymer | Xanthan Gum | 0.1 |
| MONATERIC | Surfactant | Disodium | 8.0 |
| 949J | | Lauroamphodiacetate | |
| TEGOBETAINE | Surfactant | Cocamidopropyl Betaine | 7.0 |
| L7 | | | |
| RHODACAL A- | Surfactant | Sodium C₁₄-C₁₆ Olefin | 6.0 |
| 246/L | | Sulphonate | |
| GLYCEROX 767 | Emollient | PEG-6 Caprylic/Capric | 1.0 |
| | | Glyceride | |
| ARLAMOL E | Emulsifier | PPG-15 Stearyl Ether | 1.0 |
| Hexylene Glycol | Solvent | Hexylene Glycol | 1.0 |
| PHENONIP | Preservative | Phenoxyethanol + Parabens | 1.0 |
| TWEEN 20 | Viscosity | Polysorbate 20 | 2.0 |
| | adjuster | | |
| AQUAFLEUR | Fragrance | Aqua Fleur | 0.3 |
| Mod A | | | |
| ARLASOVE 200 | Solubilizer | Isoceteth-20 | 0.6 |
| Citric Acid | PH adjuster | Citric Acid (20% solution) | 7.0 |

To a main batch vessel was added the water and Disodium EDTA. The vessel was heated to 40-45° C. The vessel was stirred until the EDTA dissolved. In a separate vessel, the RHODICARE T and the Glycerin 917 were mixed until the RHODICARE dissolved. The RHODICARE/Glycerin mixture was then added to the main batch and stirred for 30 minutes. The MONATERIC 949J, the TEGOBETAINE L7, and the RHODACAL A-246/L were then added to the main batch and stirred until dissolved. The batch was cooled. The GLYCEROX 767, ARLAMOL E, ARLASOVE, and Hexylene Glycol were added during cooling and stirred until dispersed. The PHENONIP and the TWEEN 20 was then added to the main batch and stirred until dispersed. The pH was measured and adjusted to 6.45 using the citric acid solution. The product was clear. Sub-samples were taken and stored at 4° C., 40° C., and 50° C. The samples were checked over time to see if they were physically stable. The samples remained clear and none of the samples phase separated after one month storage at the specified temperatures.

### EXAMPLE 3

### Fragranced Foaming Facial Cleanser

The materials listed in Table 3 were combined as described below to generate a fragranced foaming facial cleanser.

TABLE 3

| Trade Name | Function | INCI Name | Weight % |
|---|---|---|---|
| Water | Vehicle | Water | 67.4 |
| Disodium EDTA | Chelating | Disodium EDTA | 0.1 |
| | agent | | |
| Glycerin 917 | Humectant | Glycerin 99.7% | 3.0 |
| RHODICARE T | Polymer | Xanthan Gum | 0.1 |
| MONATERIC | Surfactant | Disodium | 8.0 |
| 949J | | Lauroamphodiacetate | |
| TEGOBETAINE | Surfactant | Cocamidopropyl Betaine | 7.0 |
| L7 | | | |
| RHODACAL A- | Surfactant | Sodium C₁₄-C₁₆ Olefin | 6.0 |
| 246/L | | Sulphonate | |
| GLYCEROX 767 | Emollient | PEG-6 Caprylic/Capric | 1.0 |
| | | Glyceride | |
| ARLAMOL E | Emulsifier | PPG-15 Stearyl Ether | 1.0 |
| Hexylene Glycol | Solvent | Hexylene Glycol | 1.0 |
| PHENONIP | Preservative | Phenoxyethanol + Parabens | 1.0 |
| TWEEN 20 | Viscosity | Polysorbate 20 | 2.0 |
| | adjuster | | |
| AQUAFLEUR | Fragrance | Aqua Fleur | 0.3 |
| Mod A | | | |
| ARLASOVE 200 | Solubilizer | Isoceteth-20 | 0.7 |
| Citric Acid | PH adjuster | Citric Acid (20% solution) | 1.4 |

To a main batch vessel was added the water and Disodium EDTA. The vessel was heated to 40-45° C. The vessel was stirred until the EDTA dissolved. In a separate vessel, the RHODICARE T and the Glycerin 917 were mixed until the RHODICARE dissolved. The RHODICARE/Glycerin mixture was then added to the main batch and stirred for 30 minutes. The MONATERIC 949J, the TEGOBETAINE L7, and the RHODACAL A-246/L were then added to the main batch and stirred until dissolved. The batch was cooled. The GLYCEROX 767, ARLAMOL E, ARLASOVE, and Hexylene Glycol were added during cooling and stirred until dispersed. The PHENONIP and the TWEEN 20 was then added to the main batch and stirred until dispersed. The pH was measured and adjusted to 6.5 using the citric acid solution. The product was clear. Sub-samples were taken and stored at 4° C., 40° C., and 50° C. The samples were checked over time to see if they were physically stable. The samples remained clear and none of the samples phase separated after one month storage at the specified temperatures.

### EXAMPLE 4

### Fragranced Foaming Facial Cleanser

The materials listed in Table 4 were combined as described below to generate a fragranced foaming facial cleanser.

TABLE 4

| Trade Name | Function | INCI Name | Weight % |
|---|---|---|---|
| Water | Vehicle | Water | 68.3 |
| Disodium EDTA | Chelating | Disodium EDTA | 0.1 |
| | agent | | |
| Glycerin 917 | Humectant | Glycerin 99.7% | 3.0 |
| RHODICARE T | Polymer | Xanthan Gum | 0.1 |
| MONATERIC | Surfactant | Disodium | 8.0 |
| 949J | | Lauroamphodiacetate | |
| TEGOBETAINE | Surfactant | Cocamidopropyl Betaine | 7.0 |
| L7 | | | |
| RHODACAL A- | Surfactant | Sodium C₁₄-C₁₆ Olefin | 6.0 |
| 246/L | | Sulphonate | |
| GLYCEROX 767 | Emollient | PEG-6 Caprylic/Capric | 1.0 |
| | | Glyceride | |
| ARLAMOL E | Emulsifier | PPG-15 Stearyl Ether | 0.5 |
| Hexylene Glycol | Solvent | Hexylene Glycol | 1.0 |
| PHENONIP | Preservative | Phenoxyethanol + Parabens | 1.0 |
| TWEEN 20 | Viscosity | Polysorbate 20 | 2.0 |
| | adjuster | | |
| AQUAFLEUR | Fragrance | Aqua Fleur | 0.3 |
| Mod A | | | |
| ARLASOVE 200 | Solubilizer | Isoceteth-20 | 0.3 |
| Citric Acid | PH adjuster | Citric Acid (20% solution) | 1.4 |

To a main batch vessel was added the water and Disodium EDTA. The vessel was heated to 40-45° C. The vessel was stirred until the EDTA dissolved. In a separate vessel, the RHODICARE T and the Glycerin 917 were mixed until the RHODICARE dissolved. The RHODICARE/Glycerin mixture was then added to the main batch and stirred for 30 minutes. The MONATERIC 949J, the TEGOBETAINE L7, and the RHODACAL A-246/L were then added to the main batch and stirred until dissolved. The batch was cooled. The GLYCEROX 767, ARLAMOL E, ARLASOVE, and Hexylene Glycol were added during cooling and stirred until dispersed. The PHENONIP and the TWEEN 20 was then added to the main batch and stirred until dispersed. The pH was measured and adjusted to 6.45 using the citric acid solution. The product was clear. Sub-samples were taken and stored at 4° C., 40° C., and 50° C. The samples were checked over time to see if they were physically stable. The samples remained clear and none of the samples phase separated after one month storage at the specified temperatures.

### EXAMPLE 5

### Ether-Free Fragranced Foaming Facial Cleanser

The materials listed in Table 5 were combined as described below to generate a fragranced foaming facial cleanser.

TABLE 5

| Trade Name | Function | INCI Name | Weight % |
|---|---|---|---|
| Water | Vehicle | Water | 65.9 |
| Disodium EDTA | Chelating | Disodium EDTA | 0.1 |
| | agent | | |
| Glycerin 917 | Humectant | Glycerin 99.7% | 3.0 |
| RHODICARE T | Polymer | Xanthan Gum | 0.1 |
| MONATERIC | Surfactant | Disodium | 8.0 |
| 949J | | Lauroamphodiacetate | |
| TEGOBETAINE | Surfactant | Cocamidopropyl Betaine | 7.0 |
| L7 | | | |
| RHODACAL A- | Surfactant | Sodium C₁₄-C₁₆ Olefin | 6.0 |
| 246/L | | Sulphonate | |
| GLYCEROX 767 | Emollient | PEG-6 Caprylic/Capric | 1.0 |
| | | Glyceride | |
| CRODESTA | Emulsifier | Sucrose Cocoate | 1.0 |
| SL40 | | | |
| Hexylene Glycol | Solvent | Hexylene Glycol | 1.0 |
| PHENONIP | Preservative | Phenoxyethanol + Parabens | 1.0 |
| TWEEN 20 | Viscosity | Polysorbate 20 | 2.0 |
| | adjuster | | |
| AQUAFLEUR | Fragrance | Aqua Fleur | 0.3 |
| Mod A | | | |
| ARLASOVE 200 | Solubilizer | Isoceteth-20 | 0.9 |
| Citric Acid | PH adjuster | Citric Acid (20% solution) | 1.0 |

To a main batch vessel was added the water and Disodium EDTA. The vessel was heated to 40-45° C. The vessel was stirred until the EDTA dissolved. In a separate vessel, the RHODICARE T and the Glycerin 917 were mixed until the RHODICARE dissolved. The RHODICARE/Glycerin mixture was then added to the main batch and stirred for 30 minutes. The MONATERIC 949J, the TEGOBETAINE L7, and the RHODACAL A-246/L were then added to the main batch and stirred until dissolved. The batch was cooled. The GLYCEROX 767, CRODESTA SL40, ARLASOVE, and Hexylene Glycol were added during cooling and stirred until dispersed. The PHENONIP and the TWEEN 20 was then added to the main batch and stirred until dispersed. The pH was measured and adjusted to 6.0 using the citric acid solution. The product was slightly cloudy. Sub-samples were taken and stored at 4° C., 40° C., and 50° C. The samples were checked over time to see if they were physically stable. The samples phase separated after one month storage at each of the specified temperatures.

### EXAMPLE 6

### Consumer Testing of Formulation of Example 3 Formula

One hundred and twelve women ages 13 to 24 used both the formulation prepared in accordance with Example 3 as well as “PURE ZONE” cleanser available from L'Oreal Company on their faces for nightly cleansing.

The women used each product for a minimum of at least 3 to 7 times for a one week period. The women first poured the product onto a moistened hand or wet cloth then applied the product to their face. After rubbing the product gently on the skin, the product was rinsed therefrom with water. The women were asked questions relating to the performance of the cleansers. The results of the study are shown in Table 6 below:

TABLE 6

| Characteristic that either Completely | | |
|---|---|---|
| describes or very well describes the | | |
| product at issue | Example 3 | PURE ZONE |
| 1) Is an effective cleanser | 60 | 55 |
| 2) Efectively remove make-up and | 60 | 50 |
| cleanses at the same time | | |
| 3) Is an efective make-up remover | 66 | 39 |
| 4) Effectively removes foundation or | 53 | 43 |
| base make-up | | |
| 5) Effectively removes lipstick | 41 | 30 |
| 6) Effectively removes eye make-up | 58 | 35 |
| including mascara | | |

These numbers indicate the percentage of the mothers that indicated that the identified product possessed the given characteristic.

1. A composition comprising:
a. a water dispersible component selected from water dispersible components that when combined with at least a weight equivalent of water produces (i) a uniform clear mixture or (ii) uniform hazy mixture;
b. a liquid, water-insoluble propoxylated fatty alcohol; and
c. water.

2. The composition of claim 1 further comprising a foaming surfactant.

3. The composition of claim 2 wherein the foaming surfactant has a column height of greater than about 20 mm as determined by the Miles-Ross Test and is selected from the group consisting of non-ionic surfactants, cationic surfactants, amphoteric surfactants, anionic surfactant, and mixtures thereof.

4. The composition of claim 1 further comprising a liquid silicone.

5. The composition of claim 1 wherein the water dispersible component is selected from the group consisting of polyethylene glycol 400, hexylene glycol, propylene glycol, polypropylene glycol-10 methylglucose ether, ethoxydiglycol, polyethylene glycol-6 caprylic/capric glycerides, ethylene glycol monobutyl ether, triisopropyl citrate, polyethylene glycol-8 caprylic/capric glycerides, 3-methoxy-3-methyl-1 -butanol, dimethyl isosorbide, polyethylene-6 caprylic/capric triglyceride, and mixtures thereof.

6. The composition of claim 5 wherein the water dispersible component is selected from the group consisting of hexylene glycol, dimethyl isosorbide, polyethylene glycol-6 caprylic/capric glyceride, and mixtures thereof.

7. The composition of claim 1 wherein the liquid, water-insoluble propoxylated fatty alcohol is of the structure RO(CH₂CHOCH₃)n, wherein R is from about 12 to about 22 carbon atoms and is selected from straight chain, branched, saturated and unsaturated, and n ranges from 1 to about 100.

8. The composition of claim 1 further comprising at least one of the following:
a) polymeric emulsifer and/or a thickener;
b) a benefit agent; or
c) a nonionic emulsifier.

9. The composition of claim 8 wherein the polymeric emulsifier is polyethylene glycol-30 dipolyhydroxystearate; dimethicone copolyol; substituted acrylates; and mixtures thereof.

10. The composition of claim 8 wherein the thickener is selected from the group consisting of carbomers, acrylate copolymers, hydroxyethylcellulose modified with cetyl ether groups, polyvinylmethyl ether/maleic anhydride (PVM/MA) decadiene crosspolymer, and mixtures thereof.

11. The composition of claim 8 wherein the thickener is selected from the group consisting of acrylates/aminoacrylates copolymer, acrylates/steareth-20 methacrylate copolymer; acrylates/ceteth-20 itaconate copolymer, acrylates/steareth-20 itaconate copolymer, carbomers, modified hydroxycellulose, polyvinylacetate/maleic anhydride (PVA/MA) decadiene crosspolymer, and mixtures thereof.

12. The composition of claim 8 further comprised of a cleansing enhancer that is a nonfoaming surfactant and/or a non-ionic emulsifier.

13. The composition of claim 12 wherein the nonfoaming surfactant is selected from the group consisting of sucrose cocoate, sucrose stearate and mixtures thereof.

14. The composition of claim 12 wherein the non-ionic emulsifier is selected from the group consisting of isoceteth 20, oleth-2, mixture of PEG-40 hydrogenated castor oil and trideceth-9, Poloxamer 184, laureth-4, sorbitan trioleate, polyoxyethylene-(2) oleyl ether, sorbitan stearate, cetearyl glucoside, glyceryl oleate, and mixtures thereof.

15. The composition of claim 8 wherein the benefit agent is selected from the group consisting of vasoconstrictors, collagen enhancers, anti-edema agents, depigmentation agents; reflectants; detangling/wet combing agents; film forming polymers; humectants; amino acid agents; antimicrobial agents; allergy inhibitors; anti-acne agents; anti-aging agents; anti-wrinkling agents, antiseptics; analgesics; antitussives; antipruritics; local anesthetics; anti-hair loss agents; hair growth promoting agents; hair growth inhibitor agents; antihistamines; antiinfectives; inflammation inhibitors; anti-emetics; anticholinergics; vasoconstrictors; vasodilators; wound healing promoters; peptides, polypeptides and proteins; deodorants and anti-perspirants; medicament agents; skin emollients and skin moisturizers; skin firming agents, hair conditioners; hair softeners; hair moisturizers; vitamins; tanning agents; skin lightening agents; antifungals; depilating agents; shaving preparations; external analgesics; perfumes; counterirritants; hemorrhoidals; insecticides; poison ivy products; poison oak products; burn products; anti-diaper rash agents; prickly heat agents; make-up preparations; vitamins; amino acids and their derivatives; herbal extracts; retinoids; flavenoids; sensates; anti-oxidants; skin conditioners; hair lighteners; chelating agents; cell turnover enhancers; coloring agents; pigments; sunscreens and mixtures thereof.

16. The system of claim 8 wherein the benefit agent is selected from the group consisting of feverfew, centella asiatica, olive leaf, wheat protein, oat oil, lycopene, DMAE, soy and derivatives thereof, colloidal oatmeal, sulfonated shale oil, elubiol, 6-(1-piperidinyl)-2,4-pyrimidinediamine-3-oxide, finasteride, ketoconazole, salicylic acid, zinc pyrithione, coal tar, benzoyl peroxide, selenium sulfide, hydrocortisone, sulfur, menthol, pramoxine hydrochloride, tricetylammonium chloride, polyquaternium 10, panthenol, panthenol triacetate, vitamin A and derivatives thereof, vitamin B and derivatives thereof, vitamin C and derivatives thereof, vitamin D and derivatives thereof, vitamin E and derivatives thereof, vitamin K and derivatives thereof, keratin, lysine, arginine, hydrolyzed wheat proteins, hydrolyzed silk proteins, octyl methoxycinnamate, oxybenzone, minoxidil, titanium dioxide, zinc dioxide, retinol, erthromycin, tretinoin, and mixtures thereof.

17. A method for removing make-up from the skin and/or hair comprising applying a composition comprising applying a composition according to claim 1.

18. The method of claim 17 wherein the make-up is eye make-up.

19. The method of claim 17 wherein the make-up is mascara.

20. A foaming composition comprising, based upon the total weight of the foaming composition,
a) from about 0.1 percent to about 30percent of a water dispersible component selected from water dispersible components that when combined with at least a weight equivalent of water produces (i) a uniform clear mixture or (ii) uniform hazy mixture;
b) from about 0.1 percent to about 30 percent of a liquid, water-insoluble propoxylated fatty alcohol;
c) from about 1 percent to about 98 percent of water; and
d) from about 2 percent to about 30 percent of a foaming surfactant.
